(19) United States
(12) Patent Application Publication
(10) Pub. No.: US 2013/0166036 A1
(43) Pub. Date: Jun. 27, 2013

(54) ORTHOPEDIC DEVICE TO BE PROVIDED ON THE KNEE JOINT

(71) Applicants: André De Cortanze, Bandol (FR); Roger Lecurieux-Clerville, Marseille (FR)

(72) Inventors: André De Cortanze, Bandol (FR); Roger Lecurieux-Clerville, Marseille (FR)

(21) Appl. No.: 13/806,645

(22) PCT Filed: Jun. 17, 2011
(86) PCT No.: PCT/FR11/51389

§ 371 Date: Mar. 11, 2013

(30) Foreign Application Priority Data
Jun. 23, 2010 (FR) 1055019
Oct. 21, 2010 (FR) 1058596

(51) Int. Cl. A61F 2/38

(52) U.S. Cl. CPC A61F 2/3868
USPC 623/20.23

(57) ABSTRACT

An implantable orthopedic device for the knee, comprising at least two parts which are not connected to each other, which are capable of being positioned opposite each other, and each of which comprise a surface engaging with that of the other part in the operative position, one part is attachable onto the tibia side, and the other part is attachable on the femur side such that, according to the invention, both of said parts are to be attached onto the sides and outer side of the knee joint, one part being mobile relative to the other along a plane that is parallel to the vertical median plane of the knee joint, and onto one of the inner or outer sides of the knee.

### PRIORITY CLAIM

The present application is a National Phase entry of PCT Application No. PCT/FR2011/051389, filed Jun. 17, 2011, which claims priority from French Application No. 1055019, filed Jun. 23, 2010, and French Application No. 1058596, filed Oct. 21, 2010, the disclosures of which are hereby incorporated by reference herein in their entirety.

### FIELD OF THE INVENTION

The present invention relates to an orthopedic device intended to be fitted to a knee joint.

### BACKGROUND

Joint prostheses and devices specific to the knees require special techniques because of the complexity of the joint, in particular a knee joint must retain its freedom of movement in the vertical plane, known as the vertical median plane, while at the same time bear the weight of the body. This heavily loaded joint may suffer degradation particularly at the cartilage covering the femoral condyle and the tibial plateau as the result of a mechanical accident, as the result of wear and/or due to developing arthrosis; various types of prosthesis and device have therefore being developed over a great many years in an attempt to counter this degradation by replacing the damaged natural surfaces with artificial implants.

Thus, various prostheses and implants are known which have been covered by numerous patents and patent applications such as application EP 582514 by the Societe Implants Othopédiques Toutes Applications, published Feb. 9, 1994 and describing a total knee prosthesis, known as compound-sliding prosthesis, comprising a mechanical tibial plateau cemented into the tibia the end of which has been cut off beforehand, and a femoral element, likewise mechanical, provided with two condyles positioned symmetrically one on either side of the vertical medial plane; this femoral element, anchored in the femoral condyle which has been cut off beforehand in order to accept it, bears against the artificial tibial plateau to which it is not mechanically connected but which has concave surfaces compatible with the surfaces of the condyles that become lodged therein, thus guiding the relative movement thereof.

Various other patent applications such as application FR 2901690 by the companies Evolutis and Ortesio Implants, published on Dec. 7, 2007, or FR 2839256 by the company Science Medicine, published Nov. 7, 2003, or even FR 2932079 by Bercovy et al., published Dec. 11, 2009, also describe such total knee prostheses which ought “in theory to permit a faithful reproduction of the movement of the knee joint limiting wear and creep associated with the high and repeated loadings applied” to the elements of the prosthesis, doing so through special choices of geometric shapes, qualities of material, special arrangements, etc., to which these patents relate.

There are also implants for unicompartmental knee prostheses like the one described in patent application FR 2908040 by the company Aston Medical Developments Ltd. and Girou, published May 9, 2008 or the application FR 2812541 by Aubaniac, published Feb. 8, 2002, as well as knee joint prostheses of the “hinged” type like application FR 2935894 by the company C2F Implants, published Mar. 19, 2010.

However, all of these devices require resection of both at least one femoral condyle and part of the tibial plateau, which resection is done in a surgical intervention which is a very major operation for the patient, is invasive, and furthermore irreversible, and subject to mechanical failure (wear, rupture, cemented bond becoming uncemented, luxation, etc.) over the short- and medium-term, leading to further surgical interventions which are operations that are equally major.

Accordingly, there is a need firstly to overcome these disadvantages by avoiding such major surgical operations with complete and irreversible resections of at least one condyle of the femur and of part of the tibial plateau, and secondly to allow degradation of the joint a certain degree of natural repair.

### SUMMARY OF THE INVENTION

The present invention is an orthopedic device intended to be fitted to a knee joint, this joint comprising first and second levers consisting respectively of a femur and a tibia, of which the respective ends, namely the femoral condyle and the tibial plateau, have respective first and second bearing surfaces facing one another, the femur and the tibia being mobile in rotation relative to one another about their respective bearing surfaces and in a plane of rotation known as the sagittal plane between an extended position in which the femur and the tibia make a first angle of at most 180° between them and a flexed position in which they make a second angle of at least 20° between them.

With said joint being selectively subjected to a bearing force applied to the respective bearing surfaces of said femoral condyle and said tibial plateau, this orthopedic device comprises first and second bearing pieces that complement one another and are not directly connected together, and first and second fixing means, the first and second complementary bearing pieces having first and second respective complementary bearing surfaces; according to the invention said first and second complementary bearing pieces are fixed, when this orthopedic device is an operational state, by the first and second respective fixing means, laterally and externally, on one and the same side of the femoral condyle and of the tibial plateau so that they extend in a plane substantially parallel to said sagittal plane of rotation, and in that when the orthopedic device is in the operational state, the first and second complementary bearing surfaces are in mutual contact at least when the levers are in the extended position, namely the femur and tibia, the consequence of this being a reduction in said bearing force applied, in this extended position, to the first and second respective bearing surfaces of the ends of the levers, femur and tibia.

The contour of each of these complementary bearing pieces, represented by the projection of the shape of these pieces in a plane parallel to the plane defined by the sagittal plane of the joint, on one of the sides of which they are intended to be fixed, comprises at least one side known as the active side able to come into contact, at least when the joint is in the extended position, with that of the other piece.

In one embodiment, the first complementary bearing piece, intended to be fixed on the side of the first lever and able to bear against the piece intended to be fixed on the side of the second lever, is a sealed rolling bearing, and the second complementary bearing piece is a plate of which the active side contact surface forms a ramp over which the peripheral contact surface of the rolling bearing rolls.

The shapes and fixings of the two said complementary bearing pieces are determined so that they come into contact with and bear against one another, when the joint is being flexed, only temporarily during extension and at the start of flexing of the levers, femur and tibia, and at the same time generating a set clearance between the surfaces of the ends of the levers which face one another on the side of the device, namely the tibial plateau and the femoral condyle of the knee in question.

Such a novel orthopedic device intended to be fitted to a knee joint thus addresses the stated problem because, being fixed laterally and externally to the joint, the surgical intervention required is fairly “minor”; the surfaces facing one another, and that normally bear against one another, of the tibial plateau on one side and of the femoral condyles on the other are preserved; further, the elements of the device according to the invention that bear the bearing load at least when the knee is in the extended position and that thus relieve the joint on the side on which the device is installed, alleviate pain.

Since the invention also makes it possible to generate a set clearance between the surfaces of the tibial plateau and of the femoral condyle, it has been discovered to the great surprise of rheumatologists who consider that destroyed cartilage is rather irreversibly destroyed, that this cartilage can grow back provided, firstly, that the relevant zone is not loaded, which is something that the present device according to the invention makes it possible to achieve and, secondly, that the patient follows an associated treatment.

Thus, by operating from the side, and one after the other if necessary, it is possible to allow the natural bearing supports of the knee to grow back, and therefore remove the device, the elements of which have been fixed both on the tibia side and on the femur side, by a mechanical means, to the tibial plateau and to the femoral condyle respectively, in a way that allows for subsequent removal thereof (that can be qualified as reversible, as opposed to the irreversible nature of the devices hitherto known as described earlier) thus allowing the knee to operate normally and pain-free without any artificial device.

For preference, the device according to the invention is intended to be fixed subcutaneously and directly against the bones, without even having to cut off a lateral part of these bones, as described hereinafter, but it may also be fixed using two other techniques such as: (1) subcutaneous but with preparation of the bone to form, on the side both of the tibial plateau and of the femoral condyle in question, planes of section which are parallel to the vertical median plane and allow the pieces of the device to be fitted with significant area of contact on the surfaces thus prepared in order, as the case may be, to obtain better fixation and load spreading, and (2) to position the two pieces, which are intended to come into contact against one another, on the outside of the skin, fixing these pieces to the bone using transcutaneous fixators.

The result is a novel orthopedic device intended to be fitted to a knee joint of which the abovementioned advantages demonstrate the benefit and of which the description and FIGS. 5 to 8 appended hereto provide one concrete example.

However, other embodiments are possible within the scope of the present invention.

### BRIEF DESCRIPTION OF THE DRAWINGS

FIG. 1 depicts one theoretical basic design of a device that has certain features of the invention and is fixed to a knee joint, seen in side view facing the vertical median plane with the device extended.

FIG. 2 depicts the same device, again viewed from the side, facing the vertical median plane, but flexed.

FIG. 3 is a section through the device on the plane III, III′ of FIG. 1, which plane is perpendicular to the vertical median plane of the knee joint.

FIG. 4 depicts the two main pieces of the device according to one embodiment of the invention, arranged in a plane parallel to the vertical median plane of the joint against which they are intended to be fixed.

FIGS. 5 to 7 depict one embodiment of the device according to the invention in the same configurations as that of FIGS. 1 to 3.

FIG. 8 depict simplified views of the device according to the invention in the same configuration as that of FIG. 7 but, on the one hand, as an external rather than a sectioned view and, on the other hand, with an additional feature the detail of which is depicted in enlarged form in FIG. 8c.

### DETAILED DESCRIPTION

In the present description, the knee joint is defined as a joint comprising first and second levers 10, 18 consisting respectively of a femur 10 and of a tibia 18, of which the respective ends 6, 8, which in the case of a knee are respectively the femoral condyle and the tibial plateau, have first and second bearing surfaces 23, 24 facing one another.

These levers 10, 18 are mobile in rotation relative to one another about their respective bearing surfaces 23, 24 and in a plane of rotation known as the sagittal plane, between an extended position in which these levers 10, 18 make between them a first angle of at most 180° between the axes 13 and 21 thereof, and a flexed position in which these levers 10, 18 make a second angle of at least 20° between them, said joint 25 being selectively subjected to a bearing force applied to the respective bearing surfaces 23, 24 of the ends 6, 8 of said levers 10, 18.

The orthopedic device intended to be fitted to this joint 25, like that of a knee as depicted in the figures, thus comprises at least first and second bearing pieces 1, 7 that are complementary and not directly joined together, and first and second fixing means 2, the first and second complementary bearing pieces 1, 7 having first and second respective complementary bearing surfaces 9, 11.

Said second complementary bearing piece 7 is able to be fixed on the side of the second lever, such as the tibia 18, and said first piece 1 on the side of the first lever, such as the femur 10.

According to an embodiment of the invention, these two said first and second complementary bearing pieces 1, 7 are fixed, when this orthopedic device is in an operational state, by first and second respective fixing means 2, laterally and externally, to the knee joint, on one and the same side, inside or outside, of the respective ends 6, 8, such as the femoral condyle and the tibial plateau, of the first 10 and second 18 levers, namely the femur and the tibia respectively, so that they extend in a plane substantially parallel to said plane of rotation or sagittal plane, and in that when the orthopedic device is in the operational state, the first 9 and second 11 complementary bearing surfaces are in mutual contact at least when the levers 10, 18 are in the extended position, the consequence of this being a reduction in said bearing force applied, in this extended position, to the first and second respective bearing surfaces 23, 24 of the ends 6, 8 of said levers 10, 18, namely therefore the femoral condyle in the case of the femur and the tibial plateau in the case of the tibia.

In the example of the figures, the device has been depicted on the outside of a knee joint, which corresponds to the side of the internal ligament 15, and preferably subcutaneously (only the parts of bones and a ligament have been depicted in the figures).

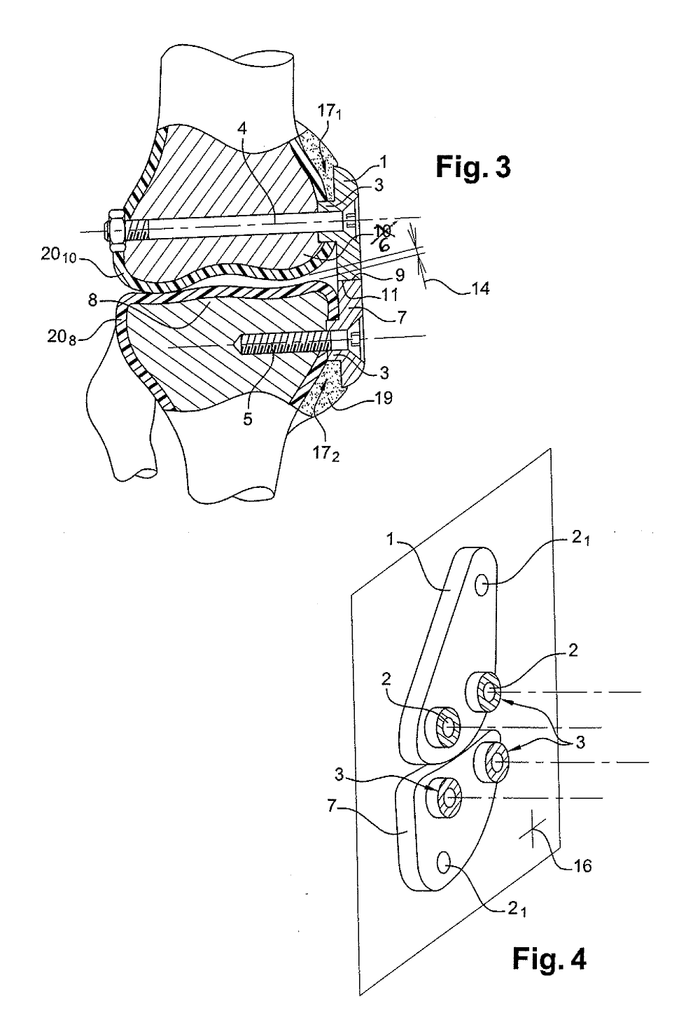

The complementary bearing pieces 1, 7 have the shape, in all of the embodiments, of plates in the case of each of which the respective surface 9, 11 that comes into contact with that of the other complementary bearing piece is produced in the thickness of the relevant piece, as depicted in FIGS. 3 and 7.

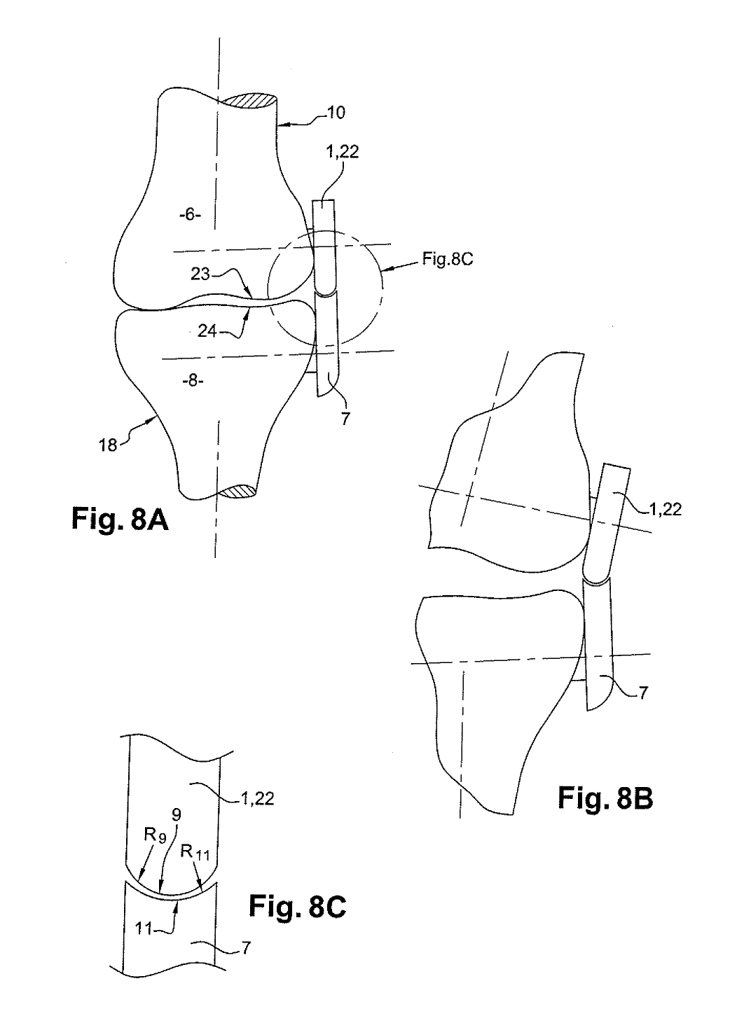

In an embodiment, as depicted in FIGS. 8A, 8B and 8C, the transverse section of the bearing surface 11 of the second complementary bearing piece 7 of the second lever 18, such as the tibia, is concave and the mating section of the bearing surface 9 of the first complementary bearing piece 1 of the first lever 10, such as the femur, is convex with a radius of curvature R₉ smaller than that R₁₁ of the concave surface 11 of the second complementary bearing piece 7 in which it engages.

Such an arrangement makes it possible to obtain a stabilizing lateral meshing effect which does not hamper physiological rotation, outside of that in the sagittal plane, of the tibia 18 with respect to the femur 10 because the mating bearing surfaces are already no longer in contact when this rotation occurs, although on the other hand this arrangement does prevent said complementary bearing pieces 1, 7 from being able to escape from one another as a result of tangential loading and of their contact surfaces which are not strictly coplanar, especially under asymmetric loading of the leg.

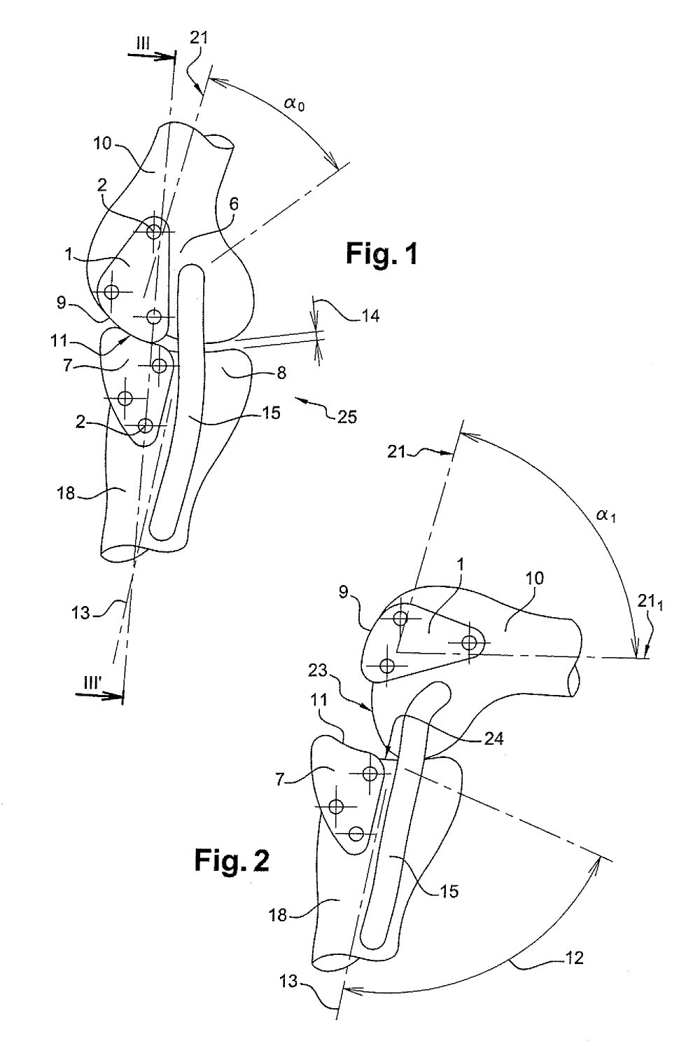

In the theoretical design as depicted in FIGS. 1 to 3, the two complementary bearing pieces 1, 7 are both plates, rigid and monobloc, immobilized with respect to the ends 6, 8 of the corresponding levers and of which the contact surface on the active side of the bearing piece 7 that is intended to be fixed to the end 8 of the second lever 18, such as the tibial plateau, forms a ramp 11 on which the contact surface of the active side of the bearing piece 1 intended to be fixed to the end 6 of the first lever 10, such as the femoral condyle, and that forms a cam 9, is able to bear.

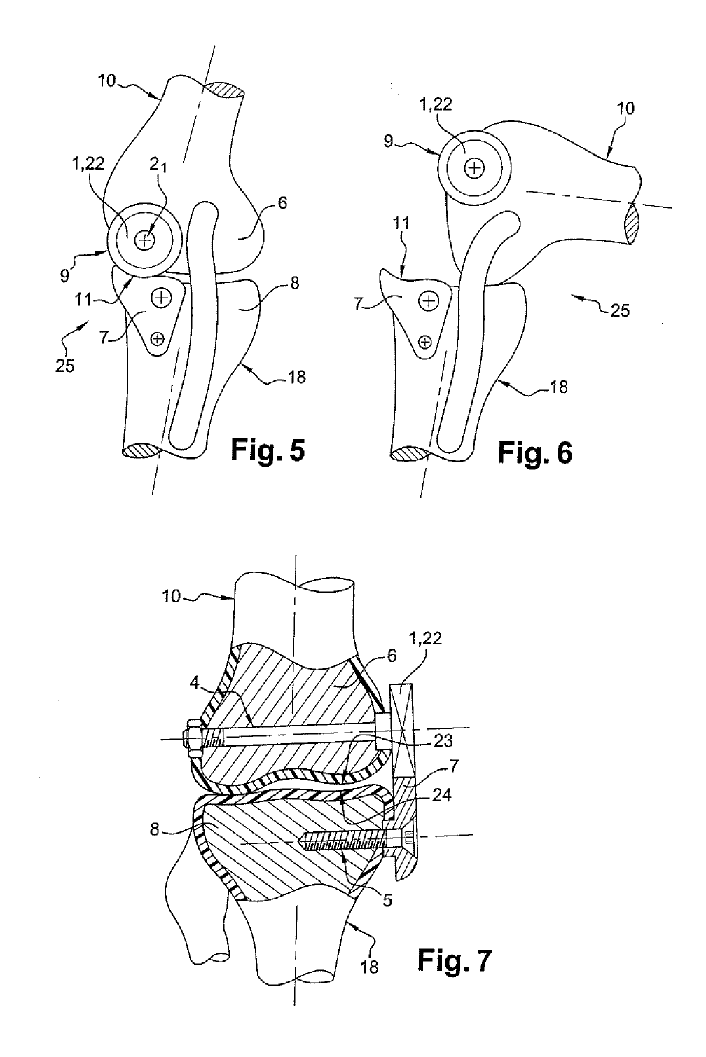

In an embodiment of the invention, which is, however, nonlimiting, other embodiments being possible, and as depicted in FIGS. 5 to 7, the first complementary bearing piece 1, intended to be fixed on the side of the first lever 10, namely the femur, is able to bear against the piece intended to be fixed on the side of the second lever 18, such as the tibial plateau, is a sealed rolling bearing 22, defined as having a rolling circular periphery, whether this be a ball bearing, a needle bearing or a roller bearing, and the second complementary bearing piece 7 is a plate as in the first embodiment above of which the active side contact surface forms a ramp 11 over which the peripheral contact surface 9 of the rolling bearing 22 rolls.

Such a ball, needle or roller type rolling bearing 22 is circular and free to rotate about its central axis 2₁ which then constitutes its only point of fixation 2.

Whatever the embodiment of this first complementary bearing piece 1 that forms a rolling bearing, there is not, in this case, unlike in the basic theoretical design, the problem of the bearing surface 9 rubbing on that 11 of the complementary second piece 7 that forms a ramp, at the start of the flexing of the joint (namely when the point of contact of the end of the first lever 10 with the end of the second lever 18, namely of the femoral condyle with the tibial plateau, does not move).

Such a rolling bearing 22 is preferably a second-generation bearing, a sealed bearing and, preferably, one with the axis incorporated into the inner race of the bearing, which inner race is doubly sealed, the entire bearing being made of biocompatible materials; the shape of said inner race, in contact with the skin, has a shape that prevents the rubbing during flexing from causing irritation.

The fixation of said complementary bearing pieces 1, 7 is defined to occur perpendicular to the vertical median plane of any joint, such as a knee, on the ends 6, 8 of the levers of which they are intended to be fixed laterally: the first and second means of fixation 2 of these the bearing pieces 1, 7 need to allow for mechanical fixation in such a way as to allow subsequent removal thereof.

Complementary bearing pieces 1, 7 each comprise one or more fixation bores 2, preferably two or three bores in the case of actual plates of the monobloc type as in FIGS. 1 to 4, one bore for the bearing pieces 1, 7 of FIGS. 5 to 7, and one bore for the rolling bearing 22 of the embodiment of FIGS. 5 to 7; at least one fixation bore 2 opening into a locating and immobilizing boss 3 of the relevant bearing piece 1, 7.

Thus, each of the complementary bearing pieces 1, 7 comprises at least one boss 3 projecting from the face of the bearing piece 1, 7 that is intended to bear laterally against the end 6, 8 of the relevant lever, namely the femur in the case of one and the tibia in the case of the other, 10, 18 and is able to be fixed by at least one bolt 4 passing through the boss 3, which bolt 4 forms the axis of rotation 2₁, in the corresponding embodiment, of the rolling bearing 22; which boss is intended to be immobilized in a hole made in the wall 20 of said end 6, 8 of the levers 10, 18, namely the cortex 20 of the corresponding bone, and sandwiching this end 6, 8, namely the tibia in the case of the one and femur in the case of the other, as depicted in FIGS. 3 and 7.

As it may be unnecessary for each bore 2 to comprise a boss 3, that potentially being dependent on the weight of the individual concerned and the bone density encountered, said complementary bearing pieces 1, 7 are able to be fixed, when they are actual plates proper, respectively to one of the sides of the ends 6, 8 of the levers 10, namely the tibia and femur, by at least one non-traversing screw 5 that screws into and becomes immobilized in the wall 20 of the end of the corresponding lever, namely the cortex 20 and in the body of these ends such as the spongy bone of the femur and of the tibia respectively, as depicted in FIGS. 3 and 7. When the complementary bearing pieces 1, 7 have at least two bosses 3 positioned relative to one another ready to be fitted as depicted in FIG. 4, they generate an installation plane 16 for the practitioner, guaranteeing correct relative positioning of said pieces.

The contour of each of these complementary bearing pieces 1, 7 which, in all cases, as indicated previously, are in the form of plates, whether these be actual plates proper or rolling bearings, represented by the projection of the shape thereof in a plane that is parallel, which may be the plane 16 defined earlier, to the one defined by the sagittal plane of the joint 25, which may be the vertical median plane of the knee, on one of the sides of which they are intended to be fixed, comprises a side known as the active side able to come into contact, at least when the joint 25 is at least in the extended position, with that of the other complementary bearing piece; the active side contact surface of the bearing piece 7 intended to be fixed to the end 8 of the second lever 18, namely the tibia side, forms a ramp 11 on which the contact surface, which is either in the shape of a cam 9 or circular in the case of a rolling bearing 22, of the active side of the bearing piece 1 intended to be fixed to the end of the first lever 10, namely the femur side, is able to bear.

By way of non-limiting indication, the dimensions of the pieces 7, 1 may be such that each can be inscribed inside a rectangular parallelepiped measuring approximately 40×45×15 mm, excluding the bosses 3 and fixation means such as screws 5 and bolts 4.

In certain embodiments, the cam 9 or rolling bearing 22 and the ramp 11 may be made of different materials, according to whether the priority is mechanical strength, friction or, on the other hand, reduction in friction, or even also device wear; the contact surfaces 9, 11 may also be lined with antifriction, plastic, composite or ceramic materials, this list not being exhaustive.

The contour of each of these two pieces 1, 7 is such that the cam 9 or rolling bearing 22 of the bearing piece 1 is able therefore to bear against the ramp 11 of the bearing piece 7 only in extension and at the start of flexing of the joint 25 on one of the sides of which they are fixed. Thus, the cam 9 or rolling bearing 22 bears temporarily against the ramp 11 during movements of the joint 25 between the extended position, in which the axis 21 of the first lever 10, such as the femur, is as vertical as possible as depicted in FIGS. 1 and 5, to a flexed angle α₀ of around 40°, between which positions this bearing makes it possible to generate some clearance 14 between the end surfaces 6, 8 of the first and second levers 10, 18, namely between the surfaces of the femoral condyle 6 of the tibial plateau 8 on the side where the device is installed, as depicted in FIGS. 1, 3, 5 and 7.

In the case of the basic theoretical design of a first complementary bearing piece which is an actual plate 1 proper, of the monobloc type, as in FIGS. 1 to 3, the profile of the cam 9 is determined according to the degree of flexing of the joint 25 during which contact between the surfaces of the ends of the first and second levers 10, 18, namely of the femoral condyle and of the tibial plateau, is to be eliminated so that cartilage regrowth can be generated in this zone which is therefore no longer loaded; this profile may have different radii of curvature in order to take account of the variety and broad spectrum of knee pathologies to be treated.

The second complementary bearing piece 7 may have a surface for contacting the ramp 11 that has different angles of attack 12 according to the normal tibial plateau/femoral condyle physiological contact resumption point chosen by the surgeon; the angle of attack 12 is defined by the angle formed by the contact surface 11 with the axis 13 of the second lever 18, which is the tibia, whereas the angle a of flexing of the first lever 10, which is the femur, is determined between the axis 21 thereof in the extended position and this same axis 21₁ in the relevant flexed position which may extend to an angle α₁ of 90 to 110°.

Between the flexed position α₀ and this extreme position α₁, the surface of the end 6, which is the femoral condyle, of the first lever 10 therefore resumes contact with the surface of the end 8, which is the tibial plateau, of the second lever 18, making it possible to spare the posterior cartilages which, in effect, need to remain in contact for that.

The surfaces for contact between either the cam 9 or the rolling bearing 22 and the ramp 11 are restricted in terms of their mobility one with respect to the other only in one degree of freedom, allowing the natural physiological rotation of the tibia with respect to the femur.

Further, the mechanical fitting/removal of the pieces of the device means that pieces within the pair such as cam 9 or rolling bearing 22 and ramp 11 can be exchanged in an attempt to keep pace with progress being made with the treatment and the controlled and progressive reduction in the clearance 14 between the femoral condyle 10 and the tibial plateau 8 as the cartilage manages to grow back; at the end of the treatment, the complete removal and ablation of these pieces leaves no sequela of joint function in the patient.

According an embodiment of the invention, and as depicted in FIG. 3, in order to obtain surface continuity between the pieces of the device, excluding of course on the side of the rolling bearing 22, which are mechanically fixed to the tibial plateau and to the femoral condyle, and the surface of the tibia and of the femur respectively, in order to ensure skin comfort, the device includes cement 19 which fills any remaining gaps 17 between the contour of said pieces 7, 1 and the lateral surface of the bones to which these pieces are fixed.

1. An orthopedic device intended to be fitted to a knee joint, this joint comprising first and second levers consisting respectively of a femur and a tibia, of which the respective ends have first and second bearing surfaces facing one another, these levers being mobile in rotation relative to one another about their respective bearing surfaces and in a plane of rotation, known as the sagittal plane, between an extended position in which these levers make a first angle of at most 180° between them and a flexed position in which these levers (10, 18) make a second angle of at least 20° between them, said joint being selectively subjected to a bearing force applied to the respective bearing surfaces of the ends of said levers, this orthopedic device comprising:
first and second bearing pieces that complement one another and are not directly connected together, and first and second fixing means, the first and second complementary bearing pieces having first and second respective complementary bearing surfaces wherein:
said first and second complementary bearing pieces are able to be fixed, when this orthopedic device is in an operational state, by the first and second respective fixing means, laterally and externally, on one and the same side of the respective ends of the first and second levers so that they are positioned in a plane substantially parallel to said plane of rotation, and when the orthopedic device is in the operational state, the first and second complementary bearing surfaces are in mutual contact at least when the levers are in the extended position, the consequence of this being a reduction in said bearing force applied, in this extended position, to the first and second respective bearing surfaces of the ends of said levers,
each of these complementary bearing pieces, has a contour represented by the projection of the shape of these pieces in a plane parallel to the plane defined by the sagittal plane of the joint, on one of the sides of which they are intended to be fixed, which contour comprises at least one side known as the active side able to come into contact, at least when the joint is in the extended position, with the contour side of the other piece,
lithe first complementary bearing piece, intended to be fixed on the side of the first lever and able to bear against the piece intended to be fixed on the side of the second lever, is a sealed rolling bearing, and the second complementary bearing piece is a plate of which the active side contact surface forms a ramp over which the peripheral contact surface of the rolling bearing rolls.

2. The device as claimed in claim 1, wherein the shapes of the two said complementary bearing pieces are determined so that these come into contact with and bear against one another, when the joint is being flexed, only temporarily during extension and at the start of flexing of this joint and at the same time generating a set clearance between the surfaces of the ends of the levers which face one another on the side of the device.

3. The device as claimed in claim 1, wherein the bearing surface of the second complementary bearing piece of the second lever has a transverse section which is concave and the bearing surface of the first complementary bearing piece of the first lever has a mating section which is convex with a radius of curvature (R₉) smaller than that (R₁₁) of the concave surface of the second complementary bearing piece in which it engages.

4. The device as claimed in claim 1, wherein the complementary bearing pieces able to be fixed laterally to the ends of the levers are mechanically fixed and in a way that allows their subsequent removal.

5. The device as claimed in claim 1, wherein at least one of the complementary bearing pieces (1, 7) comprises at least one embossing projecting from the face of the piece that is intended to bear laterally against the end of the relevant lever and is able to be fixed by at least one bolt passing through the embossing, which embossing is intended to be immobilized in a hole made in the wall of said end of lever, and sandwiching this end.

6. The device as claimed in claim 1, wherein said complementary bearing pieces are able to be fixed respectively on one of the sides of the ends of the levers by at least one non-traversing screw that becomes immobilized in the wall of the end of the corresponding lever.

7. The device as claimed in claim 1, wherein the sealed rolling bearing, which is ball, needle or roller type, is circular and free to rotate about its central axis that is the only point of fixation thereof.
